(19) United States
(12) Patent Application Publication
(10) Pub. No.: US 2020/0121550 A1
(43) Pub. Date: Apr. 23, 2020

(54) Heated Massager

(71) Applicant: Paul Elliot, Coral Springs, FL (US)

(72) Inventor: Paul Elliot, Coral Springs, FL (US)

(21) Appl. No.: 16/167,185

(22) Filed: Oct. 22, 2018

(51) Int. Cl. A61H 23/02; A61F 7/00

(52) U.S. Cl. CPC A61H 23/02; A61F 7/007; A61H 2205/083; A61H 2201/0207; A61H 2201/165

(57) ABSTRACT

A heated massager for relieving menstrual cramps includes a belt that is configured to position around and couple to a waist of a user. A vibrating and heating module is coupled to the belt so that the vibrating and heating module is selectively positionable over a lower abdomen and a lower back of a user when the belt is coupled around the waist of the user. The vibrating and heating module is configured to selectively warm and massage the lower abdomen and the lower back of the user to relieve menstrual cramps.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relate to massagers and more particularly pertains to a new massager for relieving menstrual cramps.

### BRIEF SUMMARY OF THE INVENTION

An embodiment of the disclosure meets the needs presented above by generally comprising a belt that is configured to position around and couple to a waist of a user. A vibrating and heating module is coupled to the belt so that the vibrating and heating module is selectively positionable over a lower abdomen and a lower back of a user when the belt is coupled around the waist of the user. The vibrating and heating module is configured to selectively warm and massage the lower abdomen and the lower back of the user to relieve menstrual cramps.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a heated massager according to an embodiment of the disclosure.

FIG. 2 is a bottom view of an embodiment of the disclosure.

FIG. 3 is a back view of an embodiment of the disclosure.

FIG. 4 is a cross-sectional view of an embodiment of the disclosure.

FIG. 5 is an in-use view of an embodiment of the disclosure.

### DETAILED DESCRIPTION OF THE INVENTION

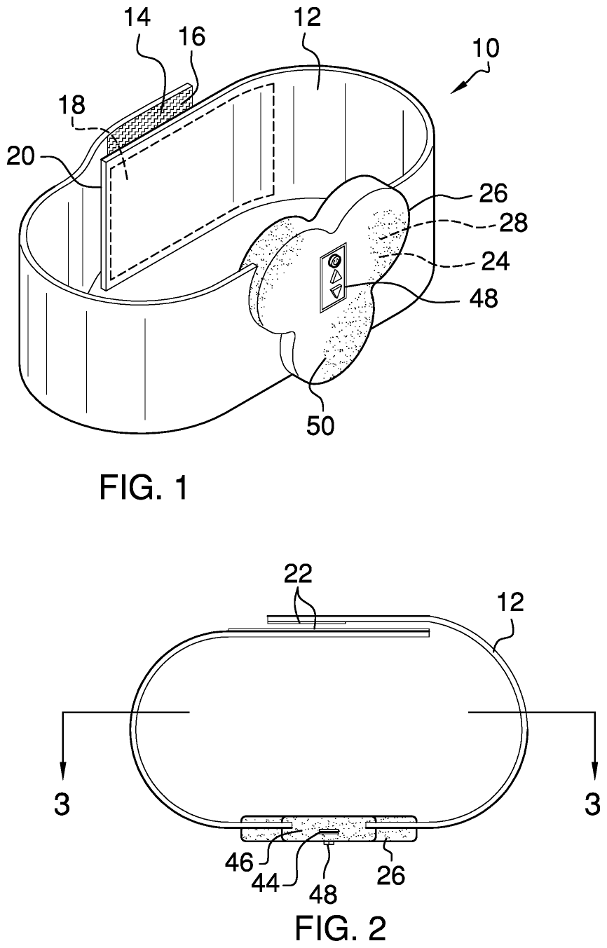

With reference now to the drawings, and in particular to FIGS. 1 through 5 thereof, a new massager embodying the principles and concepts of an embodiment of the disclosure and generally designated by the reference numeral 10 will be described.

As best illustrated in FIGS. 1 through 5, the heated massager 10 generally comprises a belt 12 that is configured to position around and couple to a waist of a user, as shown in FIG. 5. A first coupler 14 is coupled to the belt 12 proximate to a first end 16 of the belt 12. A second coupler 18 is coupled to the belt 12 proximate to a second end 20 of the belt 12. The second coupler 18 is complementary to the first coupler 14 and is positioned to selectively couple to the first coupler 14 to removably couple the belt 12 around the waist of the user. The second coupler 18 and the first coupler 14 comprises a hook and loop fastener 22, as shown in FIG. 1, to allow the belt 12 to be selectively sized to the waist of the user.

A vibrating and heating module 24 is coupled to the belt 12 so that the vibrating and heating module 24 is selectively positionable over a lower abdomen, as shown in FIG. 5, and a lower back of a user when the belt 12 is coupled around the waist of the user. The vibrating and heating module 24 is configured to selectively warm and massage the lower abdomen and the lower back of the user to relieve menstrual cramps.

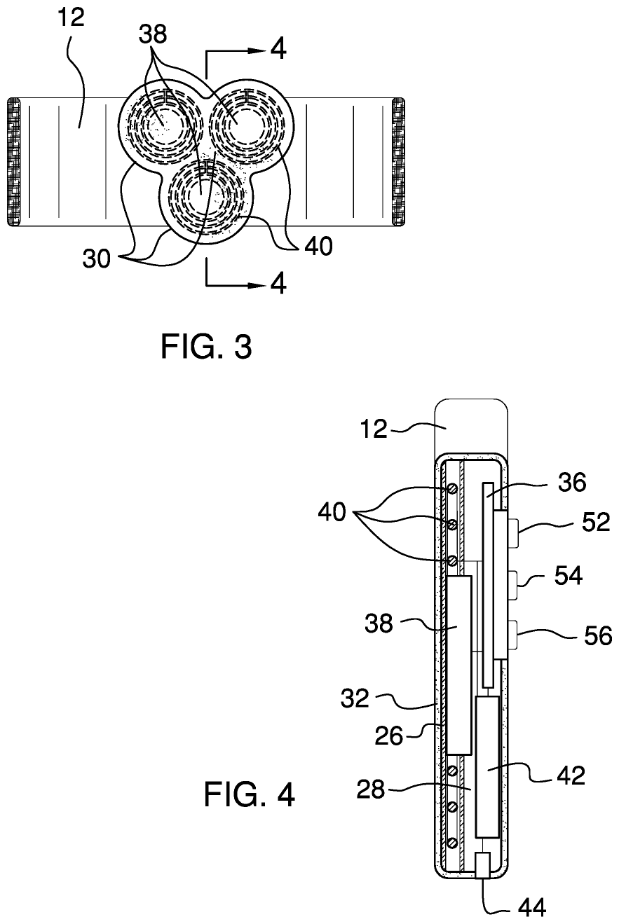

The vibrating and heating module 24 comprises a housing 26 that defines an interior space 28. The housing 26 is externally padded to rest comfortably on the lower abdomen or lower back of the user. The housing 26 comprises a plurality of fused discs 30. The plurality of fused discs 30 comprises three fused discs 30 so that the housing 26 is trilobal, as shown in FIG. 3.

A shell 32 is positioned over and is coupled to the housing 26, as shown in FIG. 3. The shell 32 comprises elastomer so that the shell 32 is resiliently compressible to rest comfortably on the lower abdomen or lower back of the user. The shell 32 comprises neoprene.

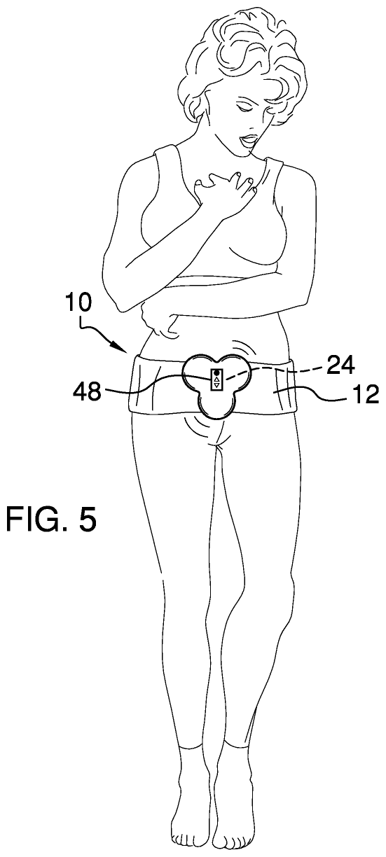

A power module 34, a microprocessor 36, a plurality of vibrators 38, and a plurality of heating elements 40 are coupled to the housing 26 and are positioned in the interior space 28, as shown in FIG. 4. The microprocessor 36 is operationally coupled to the power module 34. The vibrators 38 and the heating elements 40 are operationally coupled to the microprocessor 36. The microprocessor 36 is positioned to actuate the vibrators 38 to selectively massage the lower abdomen and the lower back of the user. The microprocessor 36 also is positioned to actuate the heating elements 40 to selectively warm the lower abdomen and the lower back of the user.

The plurality of vibrators 38 comprises vibrators 38 that are positioned singly in each fused disc 30, as shown in FIG. 3. The plurality of heating elements 40 is insulated, as shown in FIG. 4, and comprises heating elements 40 that are positioned singly in each fused disc 30, as shown in FIG. 3.

The power module 34 comprises a battery 42 that is rechargeable. A port 44 is coupled to a side edge 46 of the housing 26, as shown in FIG. 2, and is operationally coupled to the battery 42. The port 44 is configured to insert a plug of a charging cord to couple the battery 42 to a source of direct current to charge the battery 42. The port 44 is Universal Serial Bus type.

A controller 48 is coupled to a front 50 of the housing 26, as shown in FIG. 1, and is operationally coupled to the microprocessor 36. The controller 48 is positioned to input commands into the microprocessor 36 to selectively actuate the vibrators 38 and the heating elements 40.

The controller 48 comprises a first button 52, a second button 54, and a third button 56, which are selectively depressible. The first button 52 is configured to be depressed to select either the plurality of vibrators 38 or the plurality of heating elements 40 for adjustment. The second button 54 is configured to be depressed to increase the degree of vibration of the plurality of vibrators 38 or the level of heating of the plurality of heating elements 40, dependent on the selection made with the first button 52. The third button 56 is configured to be depressed to decrease the degree of vibration of the plurality of vibrators 38 or the level of heating of the plurality of heating elements 40, dependent on the selection made with the first button 52.

In use, the belt 12 is positioned around and coupled to a waist of a user with the housing 26 positioned selectively over the lower abdomen or the lower back of the user, depending on which area is to be targeted for relief of the menstrual cramps. The controller 48 is used to adjust the degree of vibration of the plurality of vibrators 38 and the level of heating of the plurality of heating elements 40, as desired by the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. A heated massager comprising:
a belt configured for positioning around and coupling to a waist of a user; and
a vibrating and heating module coupled to the belt such that the vibrating and heating module is selectively positionable over a lower abdomen and a lower back of a user when the belt is coupled around the waist of the user wherein the vibrating and heating module is configured for selectively warming and massaging the lower abdomen and the lower back of the user for relieving menstrual cramps.

2. The massager of claim 1, further comprising:
a first coupler coupled to the belt proximate to a first end of the belt; and
a second coupler coupled to the belt proximate to a second end of the belt, the second coupler being complementary to the first coupler wherein the second coupler is positioned for selectively coupling to the first coupler for removably coupling the belt around the waist of the user.

3. The massager of claim 2, further including the second coupler and the first coupler comprising a hook and loop fastener for selectively sizing the belt to the waist of the user.

4. The massager of claim 1, further the vibrating and heating module comprising:
a housing defining an interior space, the housing being externally padded such that the housing is configured for comfortably resting on the lower abdomen and lower back of the user;
a power module coupled to the housing and positioned in the interior space;
a microprocessor coupled to the housing and positioned in the interior space, the microprocessor being operationally coupled to the power module;
a plurality of vibrators coupled to the housing and positioned in the interior space, the vibrators being operationally coupled to the microprocessor wherein the microprocessor is positioned for actuating the vibrators for selectively massaging the lower abdomen and the lower back of the user; and
a plurality of heating elements coupled to the housing and positioned in the interior space, the heating elements being insulated, the heating elements being operationally coupled to the microprocessor wherein the microprocessor is positioned for actuating the heating elements for selectively warming the lower abdomen and the lower back of the user.

5. The massager of claim 4, further including the housing comprising a plurality of fused discs.

6. The massager of claim 5, further including the plurality of fused discs comprising three fused discs such that the housing is trilobal.

7. The massager of claim 4, further including a shell positioned over and coupled to the housing, the shell comprising elastomer such that the shell is resiliently compressible wherein the shell is configured for comfortably resting on the lower abdomen and lower back of the user.

8. The massager of claim 7, further including the shell comprising neoprene.

9. The massager of claim 4, further including the power module comprising a battery.

10. The massager of claim 9, further comprising:
the battery being rechargeable; and
a port coupled to a side edge of the housing, the port being operationally coupled to the battery wherein the port is configured for inserting a plug of a charging cord for coupling the battery to a source of direct current for charging the battery.

11. The massager of claim 10, further including the port being Universal Serial Bus type.

12. The massager of claim 5, further including the plurality of vibrators comprising vibrators positioned singly in each fused disc.

13. The massager of claim 5, further including the plurality of heating elements comprising heating elements positioned singly in each fused disc.

14. The massager of claim 4, further including a controller coupled to a front of the housing, the controller being operationally coupled to the microprocessor wherein the controller is positioned for inputting commands into the microprocessor for selectively actuating the vibrators and the heating elements.

15. The massager of claim 14, further including the controller comprising a first button, a second button, and a third button, the first button, the second button, and the third button being selectively depressible wherein the first button is configured for depressing for selecting either the plurality of vibrators or the plurality of heating elements for adjustment, wherein the second button is configured for depressing for increasing the degree of vibration of the plurality of vibrators or the level of heating of the plurality of heating elements, dependent on the selection made with the first button, wherein the third button is configured for depressing for decreasing the degree of vibration of the plurality of vibrators or the level of heating of the plurality of heating elements, dependent on the selection made with the first button.

16. A heated massager comprising:
a belt configured for positioning around and coupling to a waist of a user;
a first coupler coupled to the belt proximate to a first end of the belt;
a second coupler coupled to the belt proximate to a second end of the belt, the second coupler being complementary to the first coupler wherein the second coupler is positioned for selectively coupling to the first coupler for removably coupling the belt around the waist of the user, the second coupler and the first coupler comprising a hook and loop fastener for selectively sizing the belt to the waist of the user; and
a vibrating and heating module coupled to the belt such that the vibrating and heating module is selectively positionable over a lower abdomen and a lower back of a user when the belt is coupled around the waist of the user wherein the vibrating and heating module is configured for selectively warming and massaging the lower abdomen and the lower back of the user for relieving menstrual cramps, the vibrating and heating module comprising:
a housing defining an interior space, the housing being externally padded such that the housing is configured for comfortably resting on the lower abdomen and lower back of the user, the housing comprising a plurality of fused discs, the plurality of fused discs comprising three fused discs such that the housing is trilobal,
a shell positioned over and coupled to the housing, the shell comprising elastomer such that the shell is resiliently compressible wherein the shell is configured for comfortably resting on the lower abdomen and lower back of the user, the shell comprising neoprene,
a power module coupled to the housing and positioned in the interior space, the power module comprising a battery, the battery being rechargeable,
a port coupled to a side edge of the housing, the port being operationally coupled to the battery wherein the port is configured for inserting a plug of a charging cord for coupling the battery to a source of direct current for charging the battery, the port being Universal Serial Bus type,
a microprocessor coupled to the housing and positioned in the interior space, the microprocessor being operationally coupled to the power module,
a plurality of vibrators coupled to the housing and positioned in the interior space, the vibrators being operationally coupled to the microprocessor wherein the microprocessor is positioned for actuating the vibrators for selectively massaging the lower abdomen and the lower back of the user, the plurality of vibrators comprising vibrators positioned singly in each fused disc,
a plurality of heating elements coupled to the housing and positioned in the interior space, the heating elements being insulated, the heating elements being operationally coupled to the microprocessor wherein the microprocessor is positioned for actuating the heating elements for selectively warming the lower abdomen and the lower back of the user, the plurality of heating elements comprising heating elements positioned singly in each fused disc, and
a controller coupled to a front of the housing, the controller being operationally coupled to the microprocessor wherein the controller is positioned for inputting commands into the microprocessor for selectively actuating the vibrators and the heating elements, the controller comprising a first button, a second button, and a third button, the first button, the second button, and the third button being selectively depressible wherein the first button is configured for depressing for selecting either the plurality of vibrators or the plurality of heating elements for adjustment, wherein the second button is configured for depressing for increasing the degree of vibration of the plurality of vibrators or the level of heating of the plurality of heating elements, dependent on the selection made with the first button, wherein the third button is configured for depressing for decreasing the degree of vibration of the plurality of vibrators or the level of heating of the plurality of heating elements, dependent on the selection made with the first button.
